(19) United States
(12) Patent Application Publication
(10) Pub. No.: US 2011/0015711 A1
(43) Pub. Date: Jan. 20, 2011

(54) CONNECTION BETWEEN STIMULATION ELECTRODE AND CONDUCTION COIL

(75) Inventors: Christoph Vogt, St. Paul, MN (US); Lena Lewandrowski, Mainhausen (DE); Stefan Schibli, Frankfurt am Main (DE); Christiane Leitold, Woelfersheim (DE); René Richter, Babenhausen (DE)

Correspondence Address: DICKE, BILLIG & CZAJA, FIFTH STREET TOWERS, 100 SOUTH FIFTH STREET, SUITE 2250, MINNEAPOLIS, MN, 55402, US

(73) Assignee: W. C. HERAEUS GMBH, Hanau (DE)

(21) Appl. No.: 12/837,995

(22) Filed: Jul. 16, 2010

(30) Foreign Application Priority Data
Jul. 17, 2009 (DE) 10 2009 033 770.9

(51) Int. Cl. A61N 1/04

(52) U.S. Cl. 607/115

(57) ABSTRACT

One aspect includes a stimulation electrode including a base body. The base body encompasses a top area and an end area, and the end area encompasses a screw thread at least area by area.

### CROSS-REFERENCE TO RELATED APPLICATIONS

This Utility Patent Application claims priority to German Patent Application No. DE 10 2009 033 770.9, filed on Jul. 17, 2009, which is incorporated herein by reference. This Patent Application is also related to Utility Patent Applications filed on even date herewith, entitled “DETENT CONNECTION BETWEEN STIMULATION ELECTRODE AND CONDUCTION COIL” having Attorney Docket No. W683.105.101/P11135 US; entitled “CONNECTION ELEMENT FOR CONDUCTION COIL” having Attorney Docket No. W683.106.101/P11136 US; and entitled “CRIMP CONNECTION BETWEEN STIMULATION ELECTRODE AND CONDUCTION COIL” having Attorney Docket No. W683.107.101/P11137US

### BACKGROUND

One aspect relates to a stimulation electrode having a base body, wherein the base body encompasses a top area and an end area. One embodiment furthermore relates to a conduction coil for a medical electrode system. One embodiment furthermore relates to a medical electrode system having a conduction coil and a stimulation electrode, wherein the stimulation electrode encompasses a base body having a top area and an end area, the conduction coil encompasses a conduction area.

Stimulation electrodes as well as medical electrode systems are described in DE 10 2007 009 716 A1. Such stimulation electrodes must be connected to electric feed lines—also referred to as conduction coils. As a rule, the stimulation electrodes thereby consist of a high-melting metal, the feed lines of a metal having a lower melting temperature. These two components are often connected to one another by means of laser welding. However, due to the differences of the two metals, which are to be connected to one another, it is possible that the required mechanical stability or electric conductivity is not reached. Tears, which among others are caused by forming intermetallic phases or by the solidification behavior after the welding, can appear in the weld zone. Due to different melting temperatures, the fusion is partially insufficient. As a rule, such errors cannot be determined in a non-destructive manner, which can lead to considerable problems in the production or quality control, respectively.

For these and other reasons there is a need for the present invention.

### SUMMARY

One aspect relates to a stimulation electrode having a base body, wherein the base body encompasses a top area and an end area. According to one embodiment, provision is made for the end area to encompass a screw thread at least area by area.

### BRIEF DESCRIPTION OF THE DRAWINGS

The accompanying drawings are included to provide a further understanding of embodiments and are incorporated in and constitute a part of this specification. The drawings illustrate embodiments and together with the description serve to explain principles of embodiments. Other embodiments and many of the intended advantages of embodiments will be readily appreciated as they become better understood by reference to the following detailed description. The elements of the drawings are not necessarily to scale relative to each other. Like reference numerals designate corresponding similar parts.

Further advantages, features and details of embodiments result from the dependent claims and from the following description, in which a plurality of exemplary embodiments are described in detail with reference to the drawings. The features mentioned in the claims and in the description can thereby in each case be important for embodiments, either alone or in any combination.

FIG. 1 illustrates a stimulation electrode according to one embodiment.

FIG. 2 illustrates a conduction coil according to one embodiment.

FIG. 3 illustrates a further embodiment of the conduction coil according to one embodiment.

FIG. 4 illustrates a conduction coil having deviating loops.

FIG. 5 illustrates a conduction coil having a one-piece connection element.

FIG. 6 illustrates a one-piece connection element having deviating loops for forming the conduction coil.

FIG. 7 illustrates a further embodiment of the conduction coil.

FIG. 8 illustrates a medical electrode system formed from conduction coil and stimulation electrode.

FIG. 9 illustrates a further embodiment of the medical electrode system.

### DETAILED DESCRIPTION

In the following Detailed Description, reference is made to the accompanying drawings, which form a part hereof, and in which is shown by way of illustration specific embodiments in which the invention may be practiced. In this regard, directional terminology, such as “top,” “bottom,” “front,” “back,” “leading,” “trailing,” etc., is used with reference to the orientation of the Figure(s) being described. Because components of embodiments can be positioned in a number of different orientations, the directional terminology is used for purposes of illustration and is in no way limiting. It is to be understood that other embodiments may be utilized and structural or logical changes may be made without departing from the scope of the present invention. The following detailed description, therefore, is not to be taken in a limiting sense, and the scope of the present invention is defined by the appended claims.

It is to be understood that the features of the various exemplary embodiments described herein may be combined with each other, unless specifically noted otherwise.

One embodiment create a stimulation electrode, a conduction coil and a medical electrode system, in the case of which the mentioned disadvantages are avoided, for example, in the case of which a connection between the stimulation electrode and the conduction coil, which is stable and durable in the long run, is secured.

A stimulation electrode, a conduction coil, and a medical electrode system are proposed. Also a method for connecting a conduction coil to a stimulation electrode is furthermore proposed. Features and details, which are described in context with the stimulation electrode or the conduction coil or the method, thereby also apply in context with the medical electrode system and vice versa in each case.

For the stimulation electrode according to one embodiment, provision is made for the end area to encompass a screw thread at least area by area.

The main idea of one embodiment is that the stimulation electrode and the conduction coil, which will be described below, of the medical electrode system are connected to one another by means of a screw connection. For this purpose, a screw thread is applied to an outer surface of the end area of the stimulation electrode. The stimulation electrode and the conduction coil can be connected by means of this screw thread, without requiring a connection between the two by means of material engagement. The result is a permanent and resilient connection, which can be used, for example, when stimulation electrode and conduction coil in each case consist of materials, which encompass very different melting temperatures.

In the context of one embodiment, the term “screw thread” is understood as a contoured notch, which runs in a continuously helical manner around a cylindrical wall—on the inside or on the outside—in a wound helical curve. Provision is thereby made for the screw thread of the end area to be adapted to an external screw thread of the conduction coil, which will be described below, so that both can be connected to one another by means of screwing.

An embodiment of the stimulation electrode is characterized in that the end area is embodied in a cylindrical manner. This cylindrical embodiment provides for a simple and even introduction of the screw thread onto the stimulation electrode. In one embodiment, it turned out to be advantageous when the end area encompasses a longitudinal borehole. This borehole ensures a reduction of the weight of the stimulation electrode, which is thus embodied in a tube-like manner, at least in the end area.

In one embodiment, it furthermore turned out to be advantageous when the screw thread is an external screw thread. To design the screw thread as an external screw thread, the contoured notches of the screw thread are introduced onto the outer surface of the end area. Consequently, the connection thread, which will be described below, of the conduction coil must then be guided across this screw thread in a sleeve-like manner. This embodiment lends itself when the conduction is to encompass a diameter, which is greater than that of the end area. A good flexural stability is attained in this case with the conduction coil.

In an alternative embodiment, the screw thread is an internal screw thread. In that embodiment, it lends itself when the end area encompasses a longitudinal borehole. The screw thread, which is embodied as internal screw thread, can then be introduced into the inner wall of the longitudinal borehole. Such stimulation electrodes lend themselves when conduction coils including a diameter, which is smaller than the diameter of the end area, are to be connected to the stimulation electrode. Such conduction coils including a small diameter are highly flexible and a lower stress in the event that they are implanted into a human body in the context of a medical apparatus. Depending on the embodiment, the screw thread can cover the entire end area and/or the entire inner surface of the longitudinal borehole or only a part thereof.

A further embodiment of the stimulation electrode is characterized in that the stimulation electrode encompasses at least one from the group tantalum, niobium, titanium or platinum, and in one example, that the stimulation electrode encompasses a TaNbW alloy. The mentioned group of metals is characterized by a biocompatibility as well as by a high electric conductivity.

In a one embodiment alternative, the stimulation electrode encompasses a tantalum-niobium-tungsten alloy (TaNbW alloy including 10 weight % of niobium and 7.5 weight % of tungsten) or consists thereof. The tantalum-niobium-tungsten alloy turned out to be preferred in one embodiment as base material for the base body and the tantalum oxide layer, because its tensile strength is almost twice as high and because its specific capacitance is almost twice as high as compared to that of the often used platinum-iridium-10 alloy (PtIr10). A reduction of the losses in response to the transfer of stimulation pulses is thus possible.

The stimulation electrode according to one embodiment can encompass the valve metal tantalum or can consist thereof. A stimulation electrode, which is embodied in such a manner, can be provided with a tantalum oxide layer by means of high voltage pulses. The method used for this is also referred to as plasma-electrolytic oxidation (PEO) and is described in more detail in WO 2006/104432 A1 for niobium. In the disclosed method, a porous structure of the corresponding metal oxide is generated on the surface of the stimulation electrode by means of plasma-electrolytic oxidation. It thereby turned out to be an anomaly that the porous structure encompasses pores, which are considerably larger than it is known from the current state of the art. This thus leads to a stimulation electrode including an electrically conductive base body, wherein the base body in one embodiment encompasses tantalum and the base body is at least partially covered with a porous tantalum oxide layer, which is anodically applied by means of high voltage pulses.

In the context of one embodiment, the term “stimulation electrode” does not refer to the transition point of the electric energy according to physical definition, but also refers to the technical line of electric conductor and can, if need be, also include an encasing insulation as well as all further functional elements, which are fixedly connected to the line. For clarification purposes, the section of the stimulation electrode, which actually operates in the physical sense and which includes the transition point of the electric energy, will be referred to hereinbelow as “electrically active surface.”

One embodiment further relates to a conduction coil for a medical electrode system. Disadvantages of known conduction coils for medical electrode systems have been presented above. The same also applies to the resulting object. Provision is made according to one embodiment for the conduction coil to encompass a connection element on the end side and for the connection element to encompass a connection thread at least area by area. The conduction coil according to one embodiment is such that it can interact with the stimulation according to one embodiment like a plug/plug socket. The connection thread of the conduction coil is thereby in each case adapted to the screw thread of the stimulation electrode, so that both can be combined to form a medical electrode system. The main idea in the case of one embodiment of the disclosed conduction coil is that it is provided with a connection thread, so as to screw it to a stimulation electrode. The non-positive and/or positive connection resulting therefrom ensures a permanent fusion of conduction coil and stimulation electrode.

In a first embodiment alternative, the connection element is designed from a material block, in one example, in one piece. The connection element can thus encompass a tube-like or cylindrical shape. The cylindrical shape of a one-piece connection element lends itself when it is provided with an external screw thread and when it is to engage with the internal screw thread of the longitudinal borehole of the end area. In the alternative, the connection element can be an element, which is embodied in a sleeve-like or tube-like manner, which is provided in the interior with a connection thread. A connection element embodied in such a manner can be screwed like a sleeve onto the end area of the stimulation electrode. The connection element and the loops of the conduction coil are made from the same material. In one embodiment, the connection element is connected to the loops of the conduction coil by means of material engagement, for example, by means of laser welding. The design of the parts of the conduction coil from the same material, thus of the connection element and the loops, ensures that a permanent connection between the individual elements by means of material engagement can be established.

In an alternative embodiment, the connection element can be formed from a plurality of loops of the conduction coil. The loops of the conduction coil are arranged in a helical manner. A sleeve-like connection element can be formed from the loops by means of a connection, for example, by means of material engagement, such as laser welding, for instance. Subsequently, a connection thread can be cut into the loops, which are connected by means of material engagement. In the alternative, it is possible for the loops to be formed from a wire, the diameters of which are adapted to the notches of the screw thread. Consequently, the loops of the conduction coil act like a screw thread, so that the conduction coil can be screwed directly onto the screw thread of the stimulation electrode. A separate notching of thread turns of the connection thread into the connection element is thus not necessary.

A further embodiment of the conduction coil is characterized in that the conduction coil encompasses at least one from the group MP-35, MP-35N and DFT. In this alternative, the conduction coil can encompass a “drawn filled tube” (DFT). Such DFTs encompass two components, a bio-resistant, biocompatible and non-toxic component and a component of a material including a low electric resistance. For the most part, the bio-resistant, biocompatible and non-toxic component is embodied to protect the component of a material including a low electric resistance. Platinum, iridium or an alloy of these two materials is preferred in one embodiment. In a further embodiment, the core including a low electric resistance consists of a material from the vanadium group (5^{th} subgroup of the periodic table of the elements) or from the copper group (1^{st} subgroup of the periodic table of the elements). In one embodiment, the core of the DFT wire consists of tantalum (Ta), niobium (Nb) or gold (Au). The conduction coil can furthermore encompass MP-35 and/or MP-35N (MP35N is a protected mark of SPS Technologies, Inc.). MP35N substantially encompasses approximately 35 weight % of nickel, approximately 35 weight % of cobalt, approximately 20 weight % of chromium and approximately 10 weight % of molybdenum.

The loops of the conduction coil can be embodied as multiple coils, wherein the individual loops can be located coaxially and parallel to one another and can encompass the same outer diameter. The loops of the conduction coil can also be wound in a multifilar manner and can be provided with an electric insulation. In one embodiment, to attain an advantageous mass ratio for the conduction coil, which forms a defibrillation electrode, provision can be made for an outer diameter of this conduction coil to be at least five, six or seven times the diameter of the coil-forming wire or an intermediate value thereof. This leads to a flexible conduction coil including an advantageous outer dimension, which provides for a sufficiently large field intensity distribution in response to the defibrillation and thus for a relatively low shock energy.

One embodiment also relates to a medical electrode system including a conduction coil and a stimulation electrode, wherein the stimulation electrode encompasses a base body including a top area and an end area, the conduction coil encompasses a conduction area. Disadvantages of known medical electrode systems have been discussed above in detail. The object resulting therefrom is also mentioned above. Provision is made according to one embodiment for the end area of the stimulation electrode to encompass a screw thread at least area by area, wherein the conduction coil encompasses a connection element including a connection thread, which is introduced at least area by area, and wherein the connection element is screwed to the end area in a connection situation. It goes without saying that features and details, which have been described thereby in context with the conduction coil and the stimulation electrode, also apply in context with the medical electrode system and vice versa. The anomaly of the medical electrode system lies in the type, in which conduction coil and stimulation electrode are connected to one another. Contrary to known methods by means of material engagement, conduction coil and stimulation electrode are screwed to one another in a non-positive and/or positive manner. In this arrangement, which is also described as connection situation, the conduction coil and stimulation electrode, which are otherwise present separately, form the medical electrode system. One advantage of the medical electrode system according to one embodiment is that conduction coil and stimulation electrodes, which are designed from very different materials including differing characteristics, can be used.

The medical electrode system serves as electric connection between an electrotherapeutic, implantable apparatus, which can be a neuro stimulator, a pace maker, a defibrillator or another suitable electrotherapeutic implantable apparatus and the area in the body, which is to be treated. These areas in the body can be of the most varying type, such as a heart, for example. The medical electrode system cannot only serve to transfer therapeutic pulses, but also to transfer body and measuring signals to the implant, so that suitable therapy can specifically be performed in answer to the body signals.

The medical electrode system according to one embodiment encompasses an elongate body including a proximal and a distal end. Provision is made on the proximal end for a connection to an electrotherapeutic implantable apparatus. This can be a pace maker, a defibrillator or another suitable heart rhythm apparatus. A fastening device for securely fastening the stimulation electrode to the cardiac tissue is located at the distal end. On the one hand, this can be a so-called passive fixation, which is embodied in an anchor-shaped manner and which can thus hook into the myocardial muscle. On the other hand, it can be an active fixation, which can actively be screwed into the cardiac tissue by means of a helical screw, which can be screwed in. This helically embodied stimulation electrode can also be electrically conductive and can thus act as additional electrically active surface. The area of the medical electrode system, which is located between the proximal and distal end, can furthermore be sealed and insulated from the environment. The outer surface is coated here with silicon or a similar synthetic material. In the distal area of the medical electrode system, the sealed and insulated outer surface is interrupted by at least one electrically active surface. These electrically active surfaces are areas of the stimulation electrode, which can provide for a stimulation of the above-mentioned type in the atrium of the heart, for example.

One embodiment of the medical electrode system is characterized in that the two parts conduction coil—and/or connection element—and stimulation electrode which are connected to one another, are formed from metals including a different melting temperature from the group consisting of the elements Pt, Pd, Ag, Au, Nb, Ta, Ti, Zr, W, V, Hf, Mo, Co, Cr, Ni, Ir, Re, Ru as well as from alloys on the basis of at least one of these elements, and in one example, that the metal of the conduction coil encompasses a lower melting temperature than the metal of the stimulation electrode. It is known in the state of the art to connect stimulation electrodes and conduction coil to one another by means of welding, for example, laser welding. However, difficulties arise due to the differences, for example, in the melting temperatures of the two materials, which are to be connected to one another. For instance, tears can appear in the melting zone, which are caused by the different solidification behavior of the two used materials.

The medical electrode system according to one embodiment lends itself so as not to have to do without the use of materials including very different melting temperatures for the conduction coil on the one hand and for the stimulation electrode on the other hand. By means of the described embodiment of the conduction coil and of the stimulation electrode of the medical electrode system, the conduction coil on the one hand and the stimulation electrode on the other hand can in each case be formed from metals, which encompass very different melting temperatures.

In one embodiment alternative, the difference of the melting temperatures of the two parts—conduction coil and/or connection element and stimulation electrode—is at least 1000° C., and in one case, at least 1500° C. Another embodiment alternative is characterized in that the melting temperature of the higher melting material is at least 2400° C., and in one case, at least 2800° C. In an embodiment alternative, the conduction coil and/or the connection element can encompass MP-35, for instance, and the stimulation electrode can encompass tantalum. MP35 (approximately 35 weight % of nickel, approximately 35 weight % of cobalt, approximately 20 weight % of chromium and approximately 10 weight % of molybdenum) has a melting point of approximately 1400° C. Tantalum has a melting point of 2996° C., so that the temperature difference of the melting temperatures of the two materials is greater than 1500° C. In a further example, the stimulation electrode can be formed from Ta-10W, including a melting point of 3040° C. The conduction coil and/or the connection element is formed from a core jacket wire (DTF), wherein the core consists of tantalum and the jacket consists of MP35N. Here, the difference of the melting temperatures of the two parts is above 1500° C. as well. In a further example, a niobium-containing stimulation electrode is used as stimulation electrode. In the event that a conduction coil and/or a connection element of MP35N is used, the difference of the melting temperatures is more than 1000° C., because the melting temperature of niobium is 2468° C.

One embodiment also relates to a method for connecting a conduction coil to a stimulation electrode. The problems resulting thereby have been described above. The same also applies to the object resulting therefrom. Provision is now made according to one embodiment for the stimulation electrode to encompass an end area including a screw thread, which is introduced at least area by area, for the conduction coil to encompass a connection element including a connection thread, which is introduced at least area by area and for the method for connecting to include the step:
Screwing the connection element onto the end area.

It goes without saying that features and details, which have been described thereby in context with the stimulation electrode or the conduction coil or the medical electrode system, also apply in context with the method and vice versa. The anomaly of the method according to one embodiment is that a screw connection is chosen for connecting the conduction coil and the stimulation electrode. The conduction coil including the connection element thereby encompasses a means, which makes it possible to screw it onto the stimulation electrode.

A further embodiment alternative is characterized by the method step that a tube-like and/or cylindrical, and in one example, a one-piece connection element, is attached to loops by means of material engagement for forming the conduction coil. In this embodiment alternative, the connection element is made of one piece and is connected to the loops by means of material engagement, such as laser welding. In the alternative, the method step that a plurality of loops are welded to one another at least area by area for forming the connection element is disclosed in the context of one embodiment. To attain an additional connection between the conduction coil and the stimulation electrode, the connection thread and the screw thread can be adhered in a further method step.

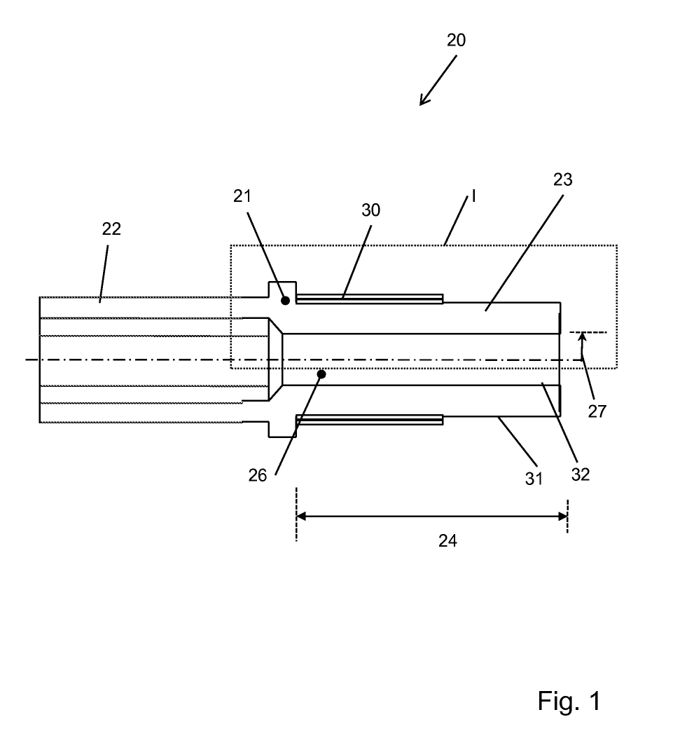

A stimulation electrode 20 according to one embodiment is illustrated in FIG. 1. This stimulation electrode 20 encompasses a base body 21, which is embodied in a tube-like manner. The base body 21 has cylinder symmetry, but is provided with a longitudinal borehole 26 in the interior. This longitudinal borehole 26 ensures a reduction of the weight of the stimulation electrode 20. The base body 21 of the stimulation electrode 20 is divided into two areas: the top area 22 on the one hand and the end area 23 on the other hand. The top area 22 encompasses the active surface, which is not illustrated in detail herein, via which electric pulses can be transferred and/or sensed. In the illustrated exemplary embodiment, a screw thread 30 is applied area by area to an outer surface 31 of the end area 23. According to one embodiment, this screw thread 30 can be applied to the entire length 24 of the end area 23 or only to an area thereof. In the alternative and not illustrated herein, it is also possible for the screw thread 30 to be introduced into an inner surface 32 of the end area 23 and to thus form an internal screw thread. The screw thread 30 illustrated herein covers the outer surface 31 of the end area only area by area and ends at a dam-like intermediate ring, which separates the top area 22 from the end area 23. At the same time, this dam-like embodiment serves as stop for a connection element 70, which will be described in detail below.

FIGS. 4 to 7 illustrate in each case an embodiment of a conduction coil 40 according to one embodiment for a medical electrode system 10, which will be described in detail below. The conduction coil 40 encompasses a conduction area 41, which includes helically arranged loops 43. The conduction coil 40 serves to conduct electric pulses from a body part, such as a heart, for instance, to a medically implantable apparatus or in opposite direction.

Conduction coils, which are welded to stimulation electrodes, are known in the state of the art. Due to a different solidification behavior, this connection by means of material engagement, however, can also encompass tears and intermetallic phases, which lead to a weakening of the connection between the conduction coil and the stimulation electrode.

To overcome this disadvantage, the conduction coil 40 according to one embodiment encompasses a connection element 70, which is arranged on the end side. This connection element 70 connects to the loops 43. For connection to the stimulation electrode 20 according to one embodiment, the conduction coil encompasses the connection element 70, which encompasses a connection thread 71 at least area by area. In the illustrated embodiment alternatives, the connection element 70 is in each case provided with an internal screw thread, so that the conduction coil 40 can be slid over the end area 23 of the stimulation electrode 20 in a sleeve-like manner and can subsequently be screwed thereto. To attain this, the connection thread 71 must be adapted to the screw thread 30 of the stimulation electrode 20. In addition, a radius 72 of the connection element 70 must be adapted to a radius 27 of the stimulation electrode 20, so as to make it possible for the conduction coil to be screwed on.

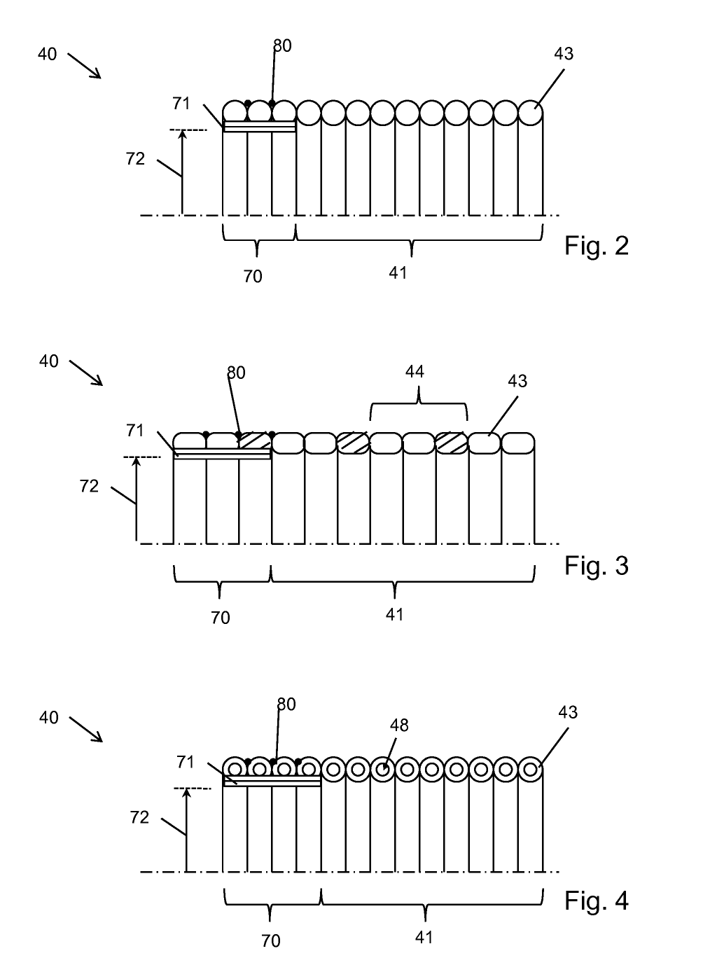

In FIGS. 2 to 4, the connection element 70 is formed from a plurality of loops 43. These loops 43 were joined to form a one-piece connection element 70 by means of a connection by means of material engagement, here by means of welding points 80. Subsequently, the internal screw thread 71 of the connection element 70 was cut into that loop 43. Depending on the embodiment of the screw thread 30 of the stimulation electrode 20, the cutting of the internal screw thread could be done without when a diameter of the loops 43 is adapted to the size of the screw thread 30 such that the loops 43 of the conduction coil 40 themselves act as connection thread. In this case, a joining of a plurality of loops 43 by means of material engagement is sufficient for forming the connection element 70. The connection thread 71 is formed by means of the loops 43.

FIG. 2 clarifies that the conduction coil 40 consists of individual loops 43, which encompass a circular cross section. To form the conduction coil and/or the loops 43 of the conduction coil, a wire is wound in a helical manner. It is also possible for the loops 43 of the conduction coil to encompass a rectangular cross section, as is clarified in FIG. 3. This cross section can be generated, for example, by pulling or crushing a wire, which is otherwise provided with a round cross section. FIG. 3 is to furthermore clarify that the conduction coil 40 can be formed by a plurality of wires. In this case, three helical parts are screwed into one another such that a conduction coil is created, in the case of which the individual loops 43 of the helical parts are arranged next to one another in a loop packet 44. This is to be clarified by means of shading, which illustrates one of the three helical parts. The three helical parts, which form the conduction coil 40, thereby form loop packets 44, which include three loops 43 in each case.

FIGS. 4 and 7 illustrate further embodiments of the conduction coil 40. The used conduction coils are so-called “drawn filled tubes” (DFT). A DFT thereby encompasses two components: a bio-resistant, non-toxic component and a component consisting of a material including a low, electric resistance. Generally, the bio-compatible component forms a cover over the component including the low electric resistance. In FIGS. 4 and 7, this is to be clarified in that each of the loops 43 encompasses a core 48, which is to represent the component including the low electric resistance. The cross section of the individual loops 43 can vary thereby, for instance with a round, rectangular or flattened cross section. Depending on the designated use, one of the illustrated types of the conduction coil 40 can be used.

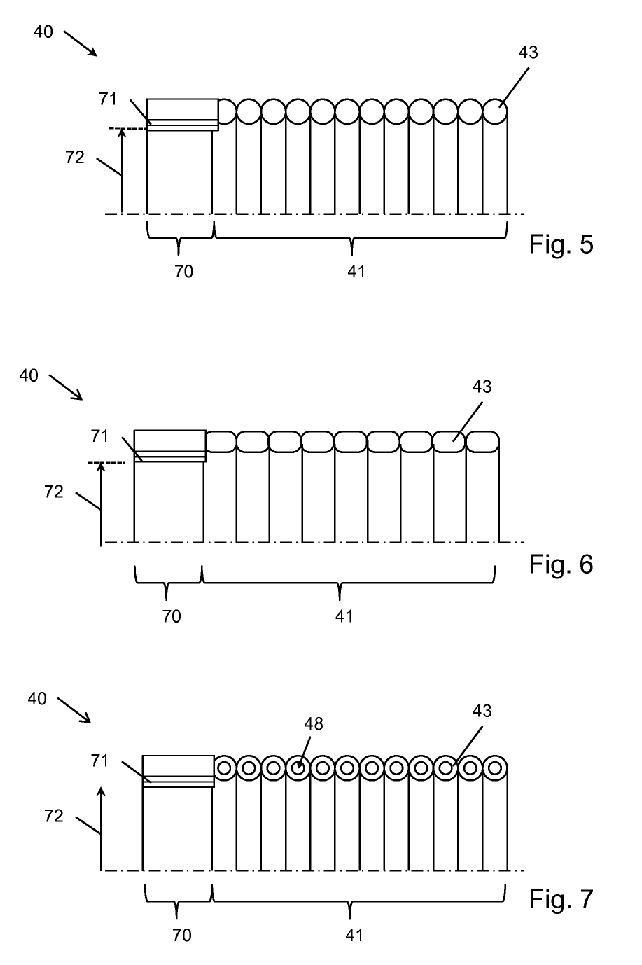

FIGS. 5 to 7 illustrate a further embodiment alternative of the conduction coil 40 according to one embodiment. In this embodiment, the connection element 70 is a tube-like, one-piece element, which is connected to the loops 43 of the conduction coil 40. In one embodiment, a connection between the connection element 70 and the loops 43 of the conduction area 41 of the conduction coil 40 by means of material engagement lends itself for this purpose. In this illustrated exemplary embodiment, an inner side of the connection element 70, which is embodied in a tube-like manner, is provided with an internal screw thread 71. This internal screw thread serves the purpose of being screwed onto the screw thread of the stimulation electrode 20 in a connection situation. This leads to a non-positive and/or positive connection between the stimulation electrode 20 and the conduction coil 40, so that the medical electrode system 10 according to one embodiment is formed as a result.

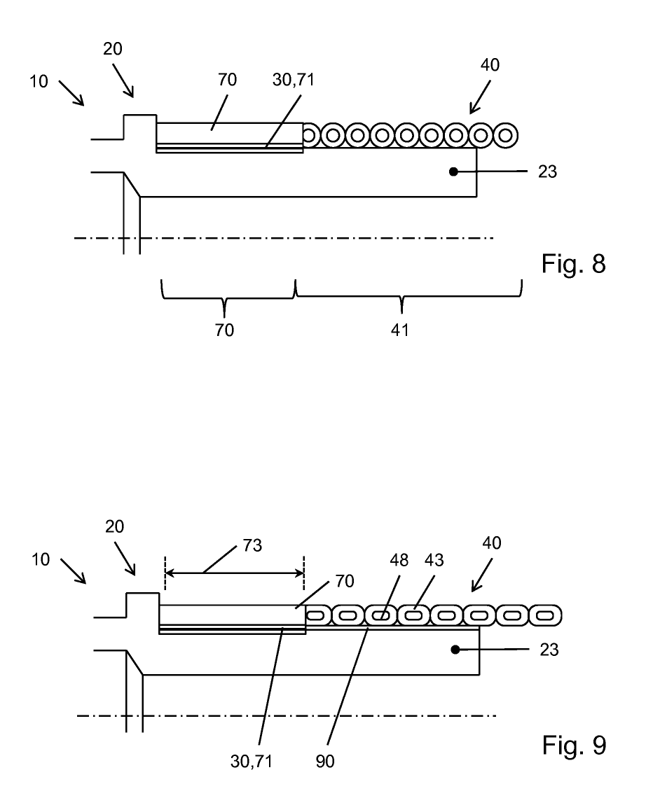

That electrode system 10 according to one embodiment is illustrated in FIGS. 8 and 9. These Figures also illustrate in each case a section through the stimulation electrode 20 according to one embodiment and the conduction coil 40 according to one embodiment. To simplify matters, only that area of the stimulation electrode 20 was drawn in, which is provided with the character I in FIG. 1. FIGS. 8 and 9 illustrate in each case a connection situation of the medical electrode system 10. Conduction coil 40 and stimulation electrode 20 are thereby connected to the connection thread 71 in a non-positive and/or positive manner via the contact of the screw thread 30. The top area of the connection element 70 thereby hits the dam-like elevation, which divides the base body into top area 22 and end area 23.

The connection element 70 drawn in FIG. 9 encompasses a length 73, which is smaller than that length 24 of the end area (see FIG. 1).

Consequently, the end area 23 can, but must not be provided with the screw thread 30 on its entire length 24. In the illustrated exemplary embodiment, the thread 30 is arranged on the end area only area by area. In the illustrated exemplary embodiment, that length of the end area 23, which is provided with the screw thread 30, equals the length 73 of the connection element 70. As a result, parts of the conduction area 41 of the conduction coil 40 still rest against the end area 23. To improve the connection between conduction coil 40 and stimulation electrode 20, an adhesive 90 can additionally be introduced between both of them. The connection element 70 can be adhered to the stimulation electrode 20 by means of the adhesive 90. In addition, some of the loops 43 of the conduction area 41 can also be adhered to the end area 23.

Although specific embodiments have been illustrated and described herein, it will be appreciated by those of ordinary skill in the art that a variety of alternate and/or equivalent implementations may be substituted for the specific embodiments shown and described without departing from the scope of the present invention. This application is intended to cover any adaptations or variations of the specific embodiments discussed herein. Therefore, it is intended that this invention be limited only by the claims and the equivalents thereof.

1. A stimulation electrode comprising:
a base body;
wherein the base body encompasses a top area and an end area;
characterized in that the end area encompasses a screw thread at least area by area.

2. The stimulation electrode according to claim 1, characterized in that the end area is embodied in a cylindrical manner, such that the end area encompasses a longitudinal borehole.

3. The stimulation electrode according to claim 1, characterized in that the screw thread is an external screw thread.

4. The stimulation electrode according to claim 1, characterized in that the screw thread is an internal screw thread.

5. The stimulation electrode according to claim 1, characterized in that the stimulation electrode encompasses at least one from the group comprising tantalum, niobium, titanium, platinum, and TaNbW alloy.

6. A conduction coil for a medical electrode system, characterized in that the conduction coil encompasses a connection element on an end side and in that the connection element encompasses a connection thread at least area by area.

7. The conduction coil according to claim 6, characterized in that the conduction coil and/or the connection element encompasses MP-35.

8. The conduction coil according to claim 6, characterized in that the loops of a conduction area of the conduction coil and the connection element are made from the same material.

9. The conduction coil according to claim 6, characterized in that the connection thread is an internal screw thread.

10. The conduction coil according to claim 6, characterized in that the connection thread is an external screw thread.

11. The conduction coil according to claim 6, characterized in that the connection element is formed from a plurality of loops of the conduction coil.

12. A medical electrode system comprising:
a conduction coil; and
a stimulation electrode;
wherein the stimulation electrode encompasses a base body comprising a top area and an end area;
wherein the conduction coil encompasses a conduction area;
characterized in that the end area of the stimulation electrode encompasses a screw thread at least area by area;
wherein the conduction coil encompasses a connection element comprising a connection thread, which is introduced at least area by area; and
wherein the connection element is screwed to the end area in a connection situation.

13. The medical electrode system according to claim 12, characterized in that the conduction coil, the connection element and the stimulation electrode are formed from metals comprising a different melting temperature from the group consisting of the elements Pt, Pd, Ag, Au, Nb, Ta, Ti, Zr, W, V, Hf, Mo, Co, Cr, Ni, Ir, Re, Ru and alloys on the basis of at least one of these elements, and that the metal of the conduction coil encompasses a lower melting temperature than the metal of the stimulation electrode.

14. The medical electrode system according to claim 12, characterized in that the conduction coil and/or the connection element and/or the stimulation electrode encompasses at least one from the group comprising MP-35, MP-35N and DFT.

15. A method for connecting a conduction coil to a stimulation electrode,
characterized in that
the stimulation electrode encompasses an end area comprising a screw thread, which is introduced at least area by area,
the conduction coil encompasses a connection element comprising a connection thread, which is introduced at least area by area,
and the method for connecting comprises the step:
screwing the connection element onto the end area.

16. The method according to claim 15, characterized in that the connection thread and the screw thread are adhered.

17. A medical electrode system comprising:
a stimulation electrode having a base body comprising a top area and an end area separated by an intermediate ring;
a screw thread configured at the end area of the stimulation electrode;
a conduction coil having a conduction area; and
a connection element comprising a connection thread configured on the conduction coil;
wherein the connection element is screwed to the screw thread thereby connecting the stimulation electrode to the conduction coil.
